(19) United States
(12) Patent Application Publication
(10) Pub. No.: US 2015/0010622 A1
(43) Pub. Date: Jan. 8, 2015

(54) CAPSULE FORMULATION COMPRISING FEXOFENADINE

(71) Applicant: R.P. Scherer Technologies, LLC, Carson City, NV (US)

(72) Inventor: Rickey S. Shelley, Largo, FL (US)

(21) Appl. No.: 14/323,042

(22) Filed: Jul. 3, 2014

Related U.S. Application Data
(60) Provisional application No. 61/842,461, filed on Jul. 3, 2013.

(51) Int. Cl. A61K 9/48; A61K 31/445

(52) U.S. Cl. CPC A61K 9/485; A61K 31/445
USPC 424/452; 514/317

(57) ABSTRACT

The present invention relates to a composition for use as a capsule fill comprising (a) fexofenadine hydrohalide; (b) alkali hydroxide, wherein the molar ratio of the alkali hydroxide to fexofenadine is greater than 1.5:1; (c) a solvent, and optionally a solubilizer. This composition is a stable solution, and comprises a sufficiently high concentration of fexofenadine to be useful for manufacture of capsules comprising a pharmaceutically efficacious amount of the fexofenadine for treatment of the symptoms of allergies, hay fever, or urticarial. Further, the present invention relates to a capsule comprising such a fill.

### FIELD OF INVENTION

The present invention generally relates to providing for a capsule formulation that comprises a fexofenadine.

### DESCRIPTION OF RELATED TECHNOLOGY

Fexofenadine is a pharmaceutically active ingredient in commercially available OTC drugs used in the treatment of the symptoms of allergies, hay fever, and urticarial. Until now, there have been no capsule formulations comprising a stable liquid fill comprising dissolved fexofenadine.

U.S. Pat. No. 6,387,400 discloses a pharmaceutical composition for use with soft gelatin capsules. This patent teaches that the fill formulations can be prepared with acidic active ingredients (col. 2, lines 40 to 44). The pharmaceutical composition allows for higher concentrations of active ingredient to be used, thus reducing overall fill volumes in softgels. The pharmaceutical composition uses a solvent system comprising polyethylene glycol and a hydroxide ion source, where the polyethylene glycol has a molecular weight of 200 to 100,000 Daltons, and the hydroxide ion source may be potassium hydroxide (col. 2, lines 45 to 54).

International Patent Publication WO 2012/159960 discloses a fexofenadine formulation in a solvent system suitable as a liquid fill composition. The solvent system comprises a mixture of at least one non-ionic hydrophilic surfactant which may be polyethylene glycol (see e.g. Example 4) and at least one non-ionic hydrophobic surfactant. The pH of the formulation may be adjusted by adding a base such as potassium hydroxide (first paragraph on page 11) to provide a pH of 4 to 9, more preferably, 5 to 6. The formulation is hypothesized to be suitable as a fill composition for soft gelatin capsules (last full paragraph on page 11).

Indian Patent No. 253721 discloses a liquid oral formulation containing fexofenadine that may be encapsulated in soft gelatin capsules. The formulation comprises fexofenadine HCl in a total amount of 18 to 35% by weight of the total liquid, one or more solubilizers in an amount from 35 to 85%, and a viscosity imparting agent selected from polyvinyl pyrrolidones such as K-30 in an amount from 7 to 18%. This patent teaches a suspension of fexofenadine HCl, but does disclose a softgel fill wherein the fexofenadine HCl is dissolved.

European Patent No. EP 1 965 768 B1 discloses a suspension formulation for fexofenadine, which comprises about 0.03% to about 4.80% of the zwitterionic dihydrate form of fexofenadine, and a co-solvent that may be polyethylene glycol-200, polyethylene glycol-300, or polyethylene glycol-400 (paragraph [0068]). The pH of the aqueous formulation is about 4.25 to about 9.43, which may be achieved using a buffer system such as a succinic acid/sodium hydroxide system. However, the amount of polyethylene glycol co-solvent employed appears to be only a minor amount (paragraph [0068]) and the suspension formulation is not intended for use in capsules (paragraph [0083]).

Oral administration of fexofenadine is highly active. One drawback of an oral composition of fexofenadine is its unpleasant and bitter taste and aftertaste. Another drawback is the fexofenadine solubility. It is therefore desirable to develop liquid filled capsules in which fexofenadine is dissolved in the fill.

### SUMMARY OF THE INVENTION

The present invention relates to a composition for use as a capsule fill comprising (a) fexofenadine hydrohalide; (b) alkali hydroxide, wherein the molar ratio of the alkali hydroxide to fexofenadine is greater than 1.5:1; and (c) a solvent. The present invention relates to a capsule comprising (1) a fill composition comprising (a) a fexofenadine hydrohalide, (b) an alkali hydroxide, wherein the molar ratio of alkali hydroxide to fexofenadine is greater than 1.5:1, and (c) a solvent; and (2) a capsule shell.

Fexofenadine is a pharmaceutically active ingredient in commercially available OTC drugs used in the treatment of the symptoms of allergies, hay fever, and urticarial. The chemical structure of fexofenadine in its neutral form is 2-[4-[1-hydroxy-4-[4-(hydroxydiphenylmethyl)-1-piperidyl]butyl]phenyl]-2-methyl-propanoic acid.

The structure of fexofenadine includes a tertiary amine group and a carboxylic group. The charge on these groups depends on the environment of the molecule, including the pH. At low pH values, both the amine group and the carboxylic group may be protonated. At high pH values, both the amine group and the carboxylic group may be unprotonated. In some instances of the intermediate pH values, the fexofenadine takes the neutral form as indicated above. In other instances, of the intermediate pH values, the fexofenadine takes a zwitterionic form of the molecule. The definition of fexofenadine also includes adducts of fexofenadine, such as salts, acids, and hydrates. The fexofenadine may be a monohydrate, a dehydrate, a trihydrate, or a tetrahydrate. The fexofenadine may be a hydrohalo acid adduct, such as HF, HCl, HBr, or HI. Under one embodiment the hydrohalo acid adduct is HCl.

Unless specifically referred to a specific crystal form, fexofenadine in the solid state of the present invention includes all known polymorphs of fexofenadine, including Forms I to VI, and VIII through XV, A1, B1, C1, X, and C.

The composition for use as a capsule fill also comprises an alkali hydroxide, such as lithium hydroxide, sodium hydroxide, and potassium hydroxide.

In addition to the active pharmaceutical ingredient fexofenadine hydrohalide and the alkali hydroxide, the composition for use as a capsule fill also comprises a solvent, such as water, ethanol, isopropanol, ethyl acetate, polyol, polyethylene glycol, and polyethylene oxide. Under one embodiment of the present invention, the solvent is a mixture of major amount of polyethylene glycol, and a minor amount of water. The fill may also optionally comprise a solubilizer, such as polyvinyl pyrrolidone.

It is one of the objects of the present invention is to provide for a capsule comprising a liquid fill comprising fexofenadine. Such a capsule preferably contains a sufficiently high concentration of fexofenadine to be pharmaceutically efficacious for treatment of the symptoms of allergies, hay fever, or urticarial. The liquid fill of such a capsule preferably is sufficiently stable for a long shelf life.

To improve the solubility of fexofenadine HCl, different molar ratios of potassium hydroxide to fexofenadine HCl were added to polyethylene glycol 300 (PEG 300) in combination with polyvinylpyrrolidone (Povidone K-90). At a 1:1 molar ratio of KOH to fexofenadine HCl, it was found that the fexofenadine HCl would initially dissolve, but over night crystals would precipitate out. However, at a 2:1 molar ratio of KOH to fexofenadine HCl, the fexofenadine HCl dissolves at a level sufficient for a pharmaceutically efficacious amount delivered in a typical softgel, and forms a stable solution. At an apparent pH of between 9.5 to 10, the fexofenadine HCl is solubilized to the extent to provide a 180 mg dose in a 1022 mg softgel.

Based on these results, it is apparent that the pH of the fill needs to be about equal to, or higher than the pKa of the amine group on the fexofenadine. Further, it is envisioned that the concentration of the [fexofenadine]:[H-fexofenadine]⁺ ratios in the fill is >0.5:1 would result in a stable fill as well. It is projected that fills with at least a pH of 9.22 are stable.

The present invention also provides for a capsule comprised of at least a shell and a fill. The fill comprises a fexofenadine hydrohalide, an alkali hydroxide, and a solvent, as described above. The capsule may be a soft capsule, such as a soft gelatin capsule, or a hard gelatin capsule. Under another embodiment of the present invention, the shell is composed of a material that does not include gelatin, such as modified starch, modified cellulose, substances derived from seaweed, and carrageenan.

### DETAILED DESCRIPTION OF THE INVENTION

The present invention generally relates to a composition for use as a capsule fill that comprises a fexofenadine, an alkali hydroxide, and a solvent. The present invention relates to a composition for use as a capsule fill comprising (a) fexofenadine hydrohalide; (b) alkali hydroxide, wherein the molar ratio of the alkali hydroxide to fexofenadine is greater than 1.5:1; and (c) a solvent.

The present invention also relates to a capsule composition comprising the fill composition as described above and a shell. The capsule comprises (1) a fill composition comprising (a) a fexofenadine hydrohalide, (b) an alkali hydroxide, wherein the molar ratio of alkali hydroxide to fexofenadine greater than 1.5:1, and (c) a solvent; and (2) a capsule shell.

For illustrative purposes, the principles of the present invention are described by referencing various exemplary embodiments. Although certain embodiments of the invention are specifically described herein, one of ordinary skill in the art will readily recognize that the same principles are equally applicable to, and can be employed in other systems and methods. Before explaining the disclosed embodiments of the present invention in detail, it is to be understood that the invention is not limited in its application to the details of any particular embodiment shown. Additionally, the terminology used herein is for the purpose of description and not of limitation. Furthermore, although certain methods are described with reference to steps that are presented herein in a certain order, in many instances, these steps may be performed in any order as may be appreciated by one skilled in the art; the novel method is therefore not limited to the particular arrangement of steps disclosed herein.

As used herein and in the appended claims, the singular forms “a”, “an”, and “the” include plural references unless the context clearly dictates otherwise. Furthermore, the terms “a” (or “an”), “one or more” and “at least one” can be used interchangeably herein. The terms “comprising”, “including”, “having” and “constructed from” can also be used interchangeably.

Fexofenadine is a pharmaceutically active ingredient in commercially available OTC drugs used in the treatment of the symptoms of allergies, hay fever, and urticarial. The chemical structure of fexofenadine in its neutral form is 2-[4-[1-hydroxy-4-[4-(hydroxydiphenylmethyl)-1-piperidyl]butyl]phenyl]-2-methyl-propanoic acid. In the alternative, the chemical structure of fexofenadine is 4-(1-hydroxy-4-(4(hydroxydiphenylmethyl)-1-piperidinyl)butyl)-alpha,alpha-dimethyl-benzeneacetic acid.

The definition of the term fexofenadine as used herein, includes the R enantiomer of 2-[4-[1-hydroxy-4-[4-(hydroxydiphenylmethyl)-1-piperidyl]butyl]phenyl]-2-methyl-propanoic acid, the S enantiomer of 2-[4-[1-hydroxy-4-[4-(hydroxydiphenylmethyl)-1-piperidyl]butyl]phenyl]-2-methyl-propanoic acid, a mixture of the R enantiomer and the S enantiomer, and a racemic mixture. The ratio of the R enantiomer to the S enantiomer may be from 1:99 to 99:1. Under one embodiment of the present invention the ratio of R enantiomer to the S enantiomer is approximately 1:1.

The structure of fexofenadine includes a tertiary amine group and a carboxylic group. The charge on these groups depends on the environment of the molecule, including the pH. At low pH values, both the amine group and the carboxylic group may be protonated. At high pH values, both the amine group and the carboxylic group may be unprotonated. In some instances of the intermediate pH values, the fexofenadine takes the neutral form as indicated above. In other instances, of the intermediate pH values, the fexofenadine takes a zwitterionic form of the molecule. The pKₐ of the amine group is about 9.53 and the pKₐ of the carboxylic group is about 4.25.

The definition of fexofenadine also includes adducts of fexofenadine, such as salts, acids, and hydrates. The fexofenadine may be a monohydrate, a dehydrate, a trihydrate, or a tetrahydrate. The fexofenadine may be a hydrohalo acid adduct, such as HF, HCl, HBr, or HI. Under one embodiment the hydrohalo acid adduct is HCl.

In instances when the fexofenadine is in its hydrohalo acid form, it is believed that the amine group on the fexofenadine is protonated, making the fexofenadine a cation, with the halide ion being the counterion.

Unless specifically referred to a specific crystal form, fexofenadine in the solid state of the present invention includes all known polymorphs of fexofenadine. Examples of fexofenadine are Forms I, II, III, IV. Examples of various crystal forms are described in U.S. Pat. Nos. 5,738,872, 5,932,247 and 5,855,912.

Form I of fexofenadine is an anhydrate, has a capillary melting point range of 196-201° C., a DSC endotherm with onset between 195 and 199° C. and a powder X-ray diffraction (“PXRD”) pattern with d-spacings of 14,89, 11.85, 7.30, 6.28, 5.91, 5.55, 5.05, 4.96, 4.85, 4.57, 4.45, 3.94, 3.89, 3.84, 3.78, 3.72, 3.63, 3.07, 3.04, 2.45 Å.

Form II of fexofenadine is a hydrate, with a capillary melting point range of 100 to 105° C., a DSC endotherm with onset between 124 and 126° C. and a PXRD pattern with d-spacings of 7.8, 6.4, 5.2, 4.9, 4.7, 4.4, 4.2, 4.1, 3.7, 3.6, 3.5 Å.

Form III of fexofenadine is an anhydrate, has a capillary melting point range of 166 to 171° C., a DSC endotherm with onset at 166° C. and a PXRD pattern with d-spacings of 8.95, 4.99, 4.88, 4.75, 4.57, 4.47, 4.46, 3.67, 3.65 Å.

Form IV of fexofenadine is a hydrate, undergoes a decomposition at 115 to 116° C., a DSC endotherm with onset at 146° C. is reported. Form IV is reported as having a PXRD pattern with d-spacings of 10.38, 6.97, 6.41, 5.55, 5.32, 5.23, 5.11, 4.98, 4.64, 4.32, 4.28, 4.12, 4.02, 3.83, 3.65, 3.51, 3.46 and 2.83 Å.

The term “hydrohalide” refers to HF, HCl, HBr, HI, or a mixture thereof. Fexofenadine hydrohalide is an hydrohalide acid adduct of fexofenadine, that under certain cases has the form [H-fexofenadine]⁺[halide]⁻.

Additional examples of polymorphs include Forms V, VI, and VIII through XV, as disclosed in U.S. Patent Application Publication No. 2003/0021849, and International Publication No. WO 2002/080857; Form XVI, as disclosed in U.S. Pat. No. 7,671,071, and Forms A1, B1, and C1, as defined in Anchal Kulshrestha and Dhaval Joshipura, 5 Der Pharma Chemica, 2013, issue 1, 279-283; Dr. Reddy's Form X as defined in U.S. Pat. No. 7,700,779; and Form C as produced or marketed by Dot Pharma (Bangalore, Karnataka, India).

The composition for use as a capsule fill also comprises an alkali hydroxide. Alkali hydroxides in their solid states are ionic solids comprising one or more elements of Group 1 cations and an OH³¹ anion. Examples of alkali hydroxides include lithium hydroxide, sodium hydroxide, potassium hydroxide, rubidium hydroxide and cesium hydroxide. Under one embodiment the alkali hydroxide is potassium hydroxide. The Group 1 cations can be a single cation, or a mixture of Group 1 cations, such as a binary mixture (for example, LiOH/NaOH, or LiOH/KOH, or NaOH/KOH), or a ternary mixture (for example, LiOH/NaOH/KOH).

In addition to the active pharmaceutical ingredient fexofenadine hydrohalide and the alkali hydroxide, the composition for use as a capsule fill also comprises a solvent. The solvent may be any pharmaceutically acceptable solvent or a mixture thereof, that is appropriate for a capsule fill. The solvent solubilizes, miscibilizes, or homogenizes most, or essentially all of the fexofenadine and any excipients. Examples of pharmaceutically acceptable solvents include water, ethanol, isopropanol, ethyl acetate, polyol, polyethylene glycol, and polyethylene oxide. The polyethylene glycol which may be used has any molecular weight at which a solution of the fexofenadine dissolves in the solution. Under one embodiment the polyethylene glycol has a molecular weight of less than 1000. Under another embodiment the polyethylene glycol has a molecular weight of less than 600.

Under one embodiment of the present invention, the solvent is a mixture of a major amount of polyethylene glycol, and a minor amount of water.

In addition to the active pharmaceutical ingredient fexofenadine, the alkali hydroxide, and a solvent, the composition for use as a capsule fill also comprises a solubilizer. An example of a solubilizer is polyvinyl pyrrolidone.

The composition of the fill may include additional ingredients. Examples of additional ingredients include excipients and active pharmaceutical ingredients. An example of an active pharmaceutical ingredient include pseudoephedrine. An example of an excipient includes a buffer.

There are many advantages of using capsules for delivery of active pharmaceutical ingredients. However, until now there were no viable softgel formulations that contain fexofenadine. This is in part due to poor solubility of many polymorphs of fexofenadine and its adducts.

One objective of the present invention is to provide for a capsule comprising a liquid fill comprising fexofenadine. Such a capsule preferably contains a sufficiently high concentration of fexofenadine to be pharmaceutically efficacious for treatment of the symptoms of allergies, hay fever, or urticarial. Under one embodiment of the present invention, the liquid fill of such a capsule is sufficiently stable for a long shelf life.

For example, the pharmaceutically efficacious amount for a fexofenadine HCl is equal to that of the commercially available tablets comprising fexofenadine HCl, namely 180 mg.

It has been found that the fexofenadine HCl is insufficiently soluble in polyethylene glycol to provide for the pharmaceutically efficacious amount of fexofenadine HCl in a reasonably size capsules.

Attempts to increase solubility of the fexofenadine included an adjustment of pH by adjusting the HCl:fexofenadine molar ratios in the polyethylene glycol (PEG 400) fill solution. However, varying the molar ratio of HCl:fexofenadine did not improve the solubility by a significant amount.

To improve the solubility of fexofenadine HCl, different molar ratios of potassium hydroxide to fexofenadine HCl were added to polyethylene glycol 300 (PEG 300) in combination with polyvinylpyrrolidone (Povidone K-90).

At a 1:1 molar ratio of KOH to fexofenadine HCl, it was found that the fexofenadine HCl would initially dissolve, but over night crystals would precipitate out. It is hypothesized that the solid form of [H-fexofenadine]⁺[Cl]⁻ salt dissolves readily, but that over time in the presence of KOH the fexofenadine takes the form of a zwitter ion, which precipitates.

At a 2:1 molar ratio of KOH to fexofenadine HCl, the fexofenadine HCl dissolves at a level sufficient for a pharmaceutically efficacious amount delivered in a typical softgel, and forms a stable solution. It is hypothesized that the amine group is converted to free amine, and the carboxylic acid group is completely deprotonated resulting in a negatively charged ion. At an apparent pH of between 9.5 to 10, the fexofenadine HCl is solubilized to the extent to provide a 180 mg dose in a 1022 mg softgel. This formulation could be encapsulated into a No. 12 oblong softgel, which is the size of softgel that is typically used for many softgel cough/cold products. The addition of the 0.87 percent Povidone K-90 also helped in solubilizing the fexofenadine HCl and protecting the shell.

Based on these results, it is apparent that the pH of the fill needs to be about equal to, or higher than the pKa of the amine group on the fexofenadine. While experimental results have indicated that the concentration of the [fexofenadine]:[H-fexofenadine]⁺ ratio of 1:1 in the fill provide for a stable fill, it is envisioned that the concentration of the [fexofenadine]:[H-fexofenadine]⁺ ratios in the fill is >0.5:1 would result in a stable fill as well. Thus, under one embodiment of the present invention, in view of Henderson-Hasselbalch equation, it is projected that fills with at least a pH of 9.22 are stable.

The phrase “about equal to” for the purposes of the use of logarithmic scales, such as pH, means ±0.303 pH units. Thus, for example, the phrase “greater than about pH 9.5” means greater than pH of 9.197.

An examplary fill formulation of a 2:1 molar ratio of KOH to fexofenadine HCl comprises 724.84 mg PEG 300, 61.32 mg of purified water, 44.90 g of KOH, 8.94 mg of Povidone K-90, and 182.00 mg of fexofenadine HCl, per each 1022 mg softgel.

The capsule fill composition, comprising a fexofenadine hydrohalide, an alkali hydroxide, and a solvent, as described above is used in preparation of a capsule. In addition to the capsule fill, the capsule also comprises a capsule shell. The shell completely surrounds the fill so as hold the fill.

The shell may be comprised of any suitable material that is known to form a capsule. Under one embodiment, the capsule shell is a soft capsule shell, such as a soft gelatin capsule shell, or a softgel shell. The shell may formed from a combination of gelatin, water, and a plasticiser. Such soft capsules may be produced by the use of a rotary die apparatus. Additional optional ingredients include an opacifier. The term “opacifier” refers to a pharmaceutically acceptable material which reduces the transparency or transmission of visible light through the system or substance to which it is added.

Under another embodiment of the present invention, the capsule is a hard gelatin capsule. The hard gelatin capsule consists of two sections, one slipping over the other, thus completely enclosing the active ingredient. The hard gelatin capsule may be formed and filled by the fill in any manner as known in the art. Under one embodiment, the hard gelatin capsule is one that is exclusively designed to optimize liquid filling.

The composition of the capsule shell is such that it is compatible with the fill. The composition of the shell should be selected to reduce the drying time and to insure the physical stability of the softgel as a result of the apparent pH of the fill being greater than 9.5.

Gelatin is a substantially pure protein food ingredient, obtained by the thermal denaturation of collagen, which is the most common structural material and most common protein in animals. Gelatin forms thermally reversible gels with water, and the gel melting temperature (<35° C.) is below that of human body temperature (37° C.), which gives gelatin products unique properties, such as reversible sol-gel transition states at near physiologic temperatures. Gelatin is an amphoteric protein with an isoionic point between 5 and 9, depending on raw material and method of manufacture. Type A gelatin, with an isoionic point of 7 to 9, is derived from collagen with acid pretreatment. Type B gelatin, with an isoionic point of 4.8 to 5.2, is the result of alkaline pretreatment of the collagen.

Examples of plasticizers include propylene glycol, glycerol, glycerin, sorbitol, and Anidrisorb.

Under another embodiment of the present invention the shell is composed of a material that does not include gelatin. Exemplary components of non-gelatin capsules include modified starch, modified cellulose, substances derived from seaweed, and carrageenan.

The shell may be composed of substances that meet the ethical, cultural, dietary, or religious restrictions of the target consumer of the capsule. Under one embodiment of the present invention, the shell meets the Kosher standards. Under another embodiment of the present invention the shell meets the Halal standards.

1. A composition for use as a capsule fill comprising
(a) fexofenadine hydrohalide;
(b) alkali hydroxide, wherein the molar ratio of the alkali hydroxide to fexofenadine is 1.5:1 or greater; and
(c) a solvent.

2. The composition of claim 1, wherein the molar ratio of the alkali hydroxide to fexofenadine is 2:1 or greater.

3. The composition of claim 1, wherein the solvent is selected from the group consisting of water, polyethylene glycol, ethanol, isopropanol, ethyl acetate, polyol, polyethylene glycol, and polyethylene oxide.

4. The composition of claim 3, wherein the solvent comprises polyethylene glycol 300 and water.

5. The composition of claim 4 further comprising a solubilizer.

6. The composition of claim 5, wherein the solubilizer is polyvinyl pyrrolidone.

7. The composition of claim 1, wherein the alkali hydroxide is potassium hydroxide.

8. The composition of claim 1, wherein the hydrohalide is HCl.

9. A composition for use as a capsule fill comprising
(a) fexofenadine hydrohalide;
(b) alkali hydroxide; and
(c) a solvent,
wherein the pH of the composition is about 9.5 or greater.

10. The composition of claim 9, wherein the hydrohalide is HCl, and alkali hydroxide is KOH.

11. The composition of claim 9, wherein the solvent comprises polyethylene glycol 300 and water.

12. The composition of claim 9, further comprising polyvinyl pyrrolidone.

13. A capsule comprising
(1) a fill composition comprising
(a) fexofenadine hydrohalide;
(b) KOH, wherein the molar ratio of KOH to fexofenadine is 1.5:1 or greater; and
(c) a solvent; and
(2) a capsule shell.

14. The capsule of claim 13, wherein the capsule shell is a soft capsule shell.
